(19) United States
(12) Patent Application Publication
(10) Pub. No.: US 2024/0036127 A1
(43) Pub. Date: Feb. 1, 2024

(54) Quadrature RF Transmit Coil At A Vertical Main Field MRI System

(71) Applicant: Zepp Europe Holding B.V., Amsterdam-Duivendrecht (NL)

(72) Inventors: Jinghua Wang, Cupertino, CA (US); Kongqiao Wang, Hefei (CN); Li Guo, Hefei (CN); Tongtong Han, Hefei (CN)

(21) Appl. No.: 17/878,726

(22) Filed: Aug. 1, 2022

(51) Int. Cl. G01R 33/34; G01R 33/48

(52) U.S. Cl. CPC G01R 33/34; G01R 33/48; A61B 5/055

(57) ABSTRACT

A radio-frequency apparatus for magnetic resonance imaging (MRI) and/or magnetic resonance spectroscopy (MRS) transmission comprising: at least one first coil resonator and at least one second coil resonator including a plurality of conductive elements, one or more of capacitive elements in each of the at least one first coil resonator and the at least one second coil resonator; the first and second coil resonators are located in a same layer of the radio-frequency apparatus with a same mode, are parallel to an axis of the subject being imaged and are electromagnetically isolated relative to each other; major components of radio-frequency fields generated by the first and second coil resonators extend in a direction that is orthogonal and perpendicular to a main magnetic field; and a combination of the first and second coil resonators are a radio-frequency apparatus to excite nuclear spins for the MRI and the MRS.

### TECHNICAL FIELD

The present teachings relate to radio-frequency quadrature transmit coils, more particularly, in a vertical B₀ field MRI/MRS system. The present teachings will also find application in a horizontal B₀ field MRI/MRS system at various main magnetic field strengths.

### BACKGROUND

Magnetic resonance imaging (MRI) is a widely used medical imaging modality. MRI technique offers numerous advantages over other imaging techniques. It has far less risk of side effects than most other imaging modalities such as radioscopy with x-rays or computed tomography (CT) or positron emission tomography (PET) because patient and medical personal are not subjected to ionizing radiation exposure in the procedure. Every year, more than 35 million MRI scans are performed in the United States and more than 70 million MRI scans are performed worldwide. Doctors often recommend MRI for the diagnoses of various diseases, such as tumors, strokes, heart problems, prostate cancer, spine diseases, etc. A high-quality scan is important for maximizing diagnostic sensitivity and making the right diagnosis. Generally, a high-quality image requires high signal to noise ratio (SNR), high contrast between normal and pathological tissues, low levels of artifact, and reasonable and acceptable spatial-temporal resolution.

In order to obtain a detectable nuclear magnetic resonance (NMR) or magnetic resonance imaging (MRI) or magnetic resonance (MR) signal, the object being imaged (also referred to herein as “object” or “subject”) must be exposed to a static basic magnetic field (usually designated as the B0 field) which is as homogeneous as possible. The basic magnetic field can be generated by a basic field magnet of the MRI system. While the magnetic resonance images are being recorded, the basic magnetic field has fast-switched gradient fields superimposed on it for spatial encoding, which are generated by gradient coils. Moreover, using radio-frequency (RF) antennas, radio-frequency pulses are radiated into the objected being imaged. RF field of these RF pulses is normally designated as B₁⁺. Using these RF pulses, the nuclear spins of the atoms in the object being imaged are excited such that the atoms are deflected by a so-called “excitation flip angle” from their equilibrium position parallel to the basic magnetic field B₀. The nuclear spins then process around the direction of the basic magnetic field B₀. The magnetic resonance signals generated in this manner are recorded by RF receiver coil. The receiver coil can be either the same coil which was used to generate the RF pulses (e.g., a transceiver coil) or a separate receive-only coil.

The performance of transmit coil is characterized by geometric coverage, uniformity of radio-frequency field, transmit efficiency and power deposition (e.g., specific absorption rate). Over past decades, several attempts have been made to design transmitting coils. Examples of which may be found in U.S. Pat. Nos. 5,543,711; 6,404,199; 6,870,453; 7,049,819; 7,233,147; 7,235,973; 7,432,709; 7,579,835; 10,175,314; 10,709,387 B2, 10,852,372; 10,912,517; and 11,047,935; Patent Application Publication Nos. 20200337644; 20200393526; International Patent Application Nos. WO2003008988A1; WO2004092760A1; WO2005071428A1; WO2013182949A1; WO2016183284; WO2019070848; and Japanese Patent No. JP4354981 the teachings of which are all incorporated by reference herein in their entirety.

Though many transmit coils have been developed for a horizontal or vertical magnetic field MRI system, there still exist the challenges in cost, field homogeneity, power deposition and transmit efficiency for different main magnetic field strengths and orientations. The present disclosure provides some of novel solutions to these challenges.

### DEFINITIONS

Unless defined otherwise, all technical and scientific terms used herein have the same meaning as commonly understood by one of ordinary skill in the art. Methods and materials similar or equivalent to those described herein can be used in the practice or testing of the present disclosure. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. The terms “optional” or “optionally” used herein mean that the subsequently described feature, event or circumstance may or may not occur, and that the description includes instances where said feature, event or circumstance occurs and instances where it does not.

Coil performance of transmit coil includes, but not limited to, uniformity of radio-frequency field, transmit efficiency and power deposition (e.g., specific absorption rate).

Image quality includes, but is not limited to, signal-to-noise ratio and its variations, contrast-to-noise and its variations, artifacts, and accuracy. Accuracy is a metric indicating the difference between an acquired image and an image as a ground truth, or a difference between a result and a “true” value.

B₁⁺ is the positive circularly polarized component of a transversal transmit field of a radio-frequency field (RF) which is generated by a transmit coil. The transmit coil can be at least one of volume coil, surface coil, one conductive element of an array coils, or a combination thereof. The transversal transmit RF field can be decomposed into two rotating fields: the positive circularly polarized component B₁⁺, which rotates in the direction of nuclear magnetic moment precession (counterclockwise direction), and the negative circularly polarized component B₁⁻, which rotates opposite to the direction of precession (clockwise direction). In an MRI/MRS system, only the positive circularly polarized component of the transmitting field B₁⁺ contributes to the excitation of proton nuclei spins, while the negative circularly polarized component of the transmitting field B₁⁻contributes to the receive sensitivity of a receiver coil. Therefore, B₁ is sometimes used herein to refer to the transmit field of a transmit coil (e.g., the RF transmit field B₁ of a transmit coil).

Either inhomogeneous transmit or inhomogeneous receiver sensitivity or both can give rise to signal and contrast inhomogeneities in the reconstructed images. Without removing or sufficiently reducing these B₁ inhomogeneities (e.g., B₁⁺ and B₁⁻ inhomogeneities), the value of MRI images in clinic and research may be compromised.

RF safety is very important at high field and ultra-high field MRI. The B1⁺ inhomogeneities may generate a local exposure where most specific absorption rate (SAR) is applied to one body region rather than the entire body. As a result, the hotspots may occur in the exposed tissues and may lead to regional damage of these tissues even when global SAR is less than US Food and Drug Administration (FDA) and International Electrotechnical Commission (IEC) SAR limits.

RF shimming, tailored RF shimming, and parallel transmission are techniques that enable high field and ultra-high field MRI at maximum image quality and RF patient safety. These techniques are based on accurate absolute phase of B₁⁺ mapping and adjust current amplitude and phase of each element of the RF coils and/or gradient configuration to maximize B₁⁺ uniformity in subsequent imaging. The estimation of transmit field is precondition of RF shimming and parallel transit techniques. RF shimming technique is coil configuration and object dependent. Thus, the transmit field must be estimated for each coil and object in RF shimming technique. Reducing time for estimating transmit field will reduce the time of applying RF shimming technique in clinical setting. Additionally, parallel transmit technique is coil configuration, object and sequence dependent. Therefore, the transmit field must be estimated for each coil, object and sequence in parallel transmit technique. Reducing time for estimating transmit field reduces the time of applying parallel transmit technique in clinical setting. The estimation of transmit field is precondition of RF shimming and parallel transit techniques.

### SUMMARY

The present teachings relate to a radio-frequency apparatus for magnetic resonance imaging (MRI) and/or magnetic resonance spectroscopy (MRS) transmission, the radio-frequency apparatus comprising (1) the first resonator including a plurality of conductive elements; (2) the second coil resonator including a plurality of conductive elements; (3) the first resonator and the second resonator are placed in the same layer of MRI/MRS system parallel to axis of the subject being imaged; (4) the first resonator and the second resonator electromagnetically isolated each other; (5) one or more of capacitive elements included in each resonator; (6) the maj or components of radio-frequency fields generated by the first resonator and the second resonator are orthogonal and perpendicular to a main magnetic field; and (7) combination of the first resonator and the second resonator as a radio-frequency apparatus to excite the nuclear spins for MRI and MRS.

The present teachings relate to a magnetic resonance imaging (MRI), the MRI comprising a radio-frequency apparatus, the radio-frequency apparatus comprising: (1) at least one first coil resonator including a plurality of conductive elements; (2) at least one second coil resonator including a plurality of conductive elements, (3) one or more of capacitive elements in each of the at least one first coil resonator and the at least one second coil resonator; (4) wherein the first coil resonator and the second coil resonator are located in a same layer of the radio-frequency apparatus, with a same mode, and the first coil resonator and the second coil resonator are parallel to an axis of the subject being imaged; (5) wherein the first coil resonator and the second coil resonator are electromagnetically isolated relative to each other; (6) wherein major components of radio-frequency fields generated by the first coil resonator and the second coil resonator extend in a direction that is orthogonal and perpendicular to a main magnetic field; and (7) wherein a combination of the first coil resonator and the second coil resonator are a radio-frequency apparatus to excite nuclear spins for the MRI.

The present teachings relate to a magnetic resonance spectroscopy (MRS), the MRS comprising a radio-frequency apparatus, the radio-frequency apparatus comprising: (1) at least one first coil resonator including a plurality of conductive elements; (2) at least one second coil resonator including a plurality of conductive elements, (3) one or more of capacitive elements in each of the at least one first coil resonator and the at least one second coil resonator; (4) wherein the first coil resonator and the second coil resonator are located in a same layer of the radio-frequency apparatus, with a same mode, and the first coil resonator and the second coil resonator are parallel to an axis of the subject being imaged; (5) wherein the first coil resonator and the second coil resonator are electromagnetically isolated relative to each other; (6) wherein maj or components of radio-frequency fields generated by the first coil resonator and the second coil resonator extend in a direction that is orthogonal and perpendicular to a main magnetic field; and (7) wherein a combination of the first coil resonator and the second coil resonator are a radio-frequency apparatus to excite nuclear spins for the MRS.

The present teachings provide, the conductive elements are comprised of one or more of ground dipole coil, slot coil, dipole coil, helical coil, spiral coil, fractal coil, and microstrip coil.

The present teachings provide, a transmit coil configuration that is optimized by maximized magnitude B₁⁺ and minimized magnitude B₁⁻. The transmit coil may have a perfectly positive and circularly polarized transmit field when its magnitude B₁⁻ is zero.

The above-described subject matter may also be implemented as a computer-controlled apparatus, a computer process, a computing system, or an article of manufacture, such as a computer-readable storage medium.

Other systems, methods, features and/or advantages will be or may become apparent to one with skill in the art upon examination of the following drawings and detailed description. All such additional systems, methods, features and/or advantages included within this description may be protected by the accompanying claims.

### BRIEF DESCRIPTION OF DRAWINGS

The disclosure is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

FIG. 1A is a diagram illustrating an example of a vertical Bo portable MRI/MRS system.

FIG. 1B is a cross-sectional view the portable MRI/MRS system including multiple quadrature transmission coils.

FIG. 2 is a block diagram of an example of a computing device of the MRI/MRS system.

FIG.3 illustrates a plurality of a quadrature transmit coil for a vertical Bo MRI/MRS system (spiral conductive wire).

FIG.4 is a simulated Bi+magnitude of the exemplary quadrature transmit coil shown in FIG. 3.

FIG.5 is an exemplary driven circuit with decoupling interfaces for the quadrature transmit coil shown in FIG.3.

FIG.6 is a plot of S parameters vs frequency of each channel of the exemplary quadrature transmit coil shown in FIG.3 driven by the circuit shown in FIG.5.

### DETAILED DESCRIPTION

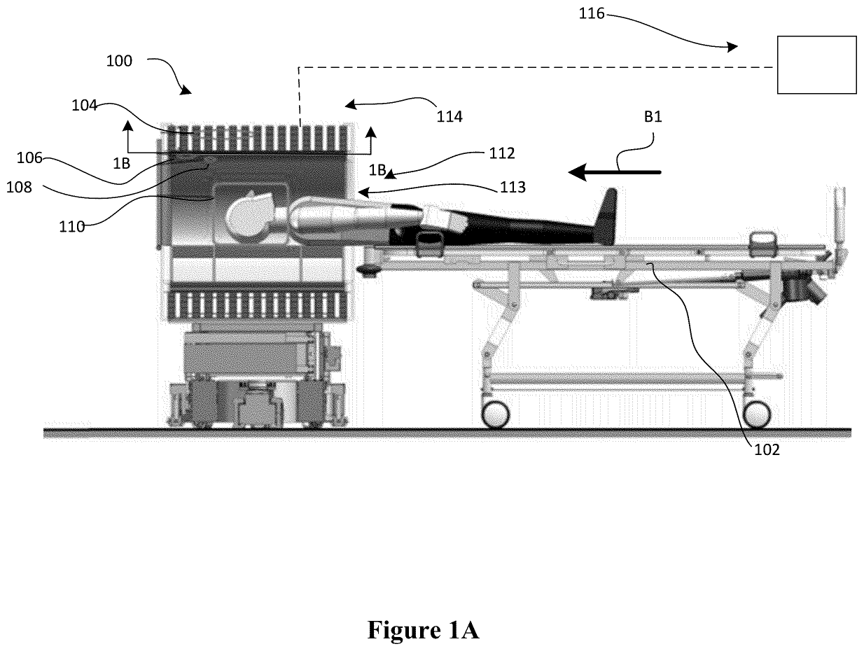

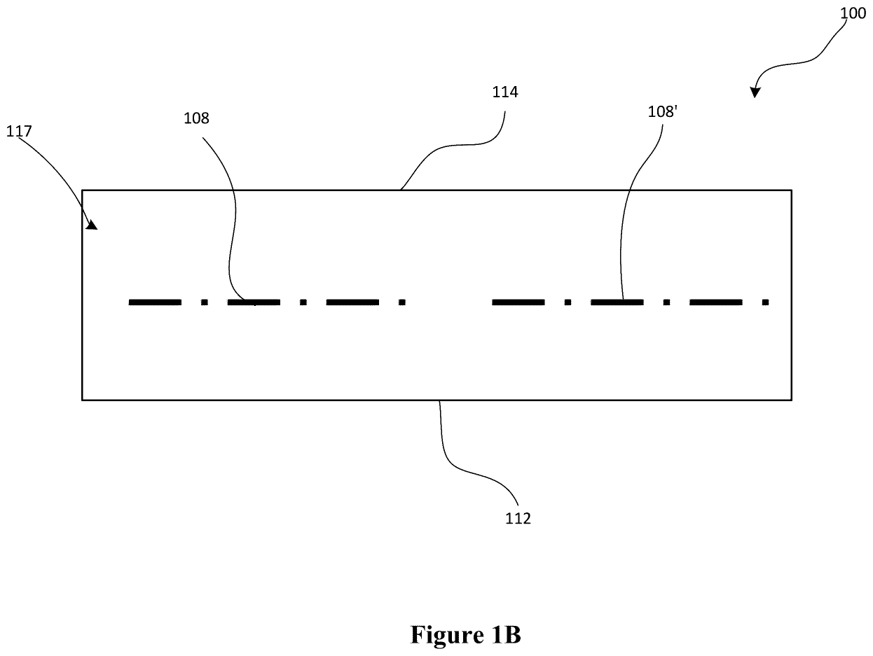

FIG. 1A is a diagram illustrating a radio-frequency apparatus (e.g., a portable MRI/MRS system) 100 with a horizontal B₁. The portable system 100 may be movable and usable with any patient table 102 or bed. The patient table may be raised or lowered to a height of the portably system 100 or the portable system 100 may be raised or lowered to a height of the patient table 102. The portable system 100 includes a permanent magnet 104. The permanent magnet 104 surrounds the patient while the patient is located in a magnet bore 113 of the permanent magnet 104. The permanent magnet 104 may work in conjunction with radio-frequency transmit coils 108 (e.g., quadrature transmission coils).

The gradient coils 106 may assist the permanent magnet 104 in creating a linear magnetic field. The magnetic field (e.g., a strong static magnetic field) may be created in any direction of an x, y, z, coordinate system for spatial encoding. The system 100 includes a radiofrequency transmission coil (RF TX coil) 108 which transmits magnetic fields excite nuclear spins for an MRI or MRS. An MRI signal reception coil (RF RX coil) 110 receives the MRI signal that is introduced by the nuclear spin precession. A plurality of k-space data is acquired by the MRI signal reception coil (RF RX coil) 110 for the portion of the subject in an imaging volume using one or more MRI sequences while the subject is located in the interior 112 of the system 100.

The radio-frequency transmit coils 108 may assist the permanent magnet 104 in creating an electromagnetic field to excite nuclear spins. A radio-frequency reception coil (RF RX Coil) 110 receives and measures the induced electromagnetic signal by the nuclear spins. The RF TX coil 108, the RF RX Coils 110, or both may operate within a radio-frequency of about 50 MHz or less, about 10 MHz or less, or about 5 MHz or less, or about 1 MHz or less. Preferably, the RF TX coil 108, the RF RX Coils 110, or both may operate within a radio-frequency of about 1 MHz to about 20 MHz.

The magnet bore 113 of the portable system 100 may be sufficiently large to fit all or a portion of a human. The magnet bore 113 may fit a torso of any individual. The cross-section of the portable system 100 may be symmetrical, asymmetrical, circular, oval, geometric, nongeometric, or a combination thereof. The magnet bore 113 of the portable system may be spaced apart from an exterior 114 by walls of the portable system 100. The magnet bore 113 may be an interior of the portable system. The magnet bore 113 may receive all or a portion of a patient. The magnet bore 113 may include a shutter that is openable or closeable. The shutter may be a plate that is moved over the removable shielding 122. A computing device 116 is connected to the portable system 100 to control the portable system and provide feedback to a user.

FIG. 1B is a cross-sectional view of a wall of the portable system 100. The wall includes an interior 112 and an exterior 114 with a cavity 117 located therein. Inside the cavity 117 is located the radio-frequency transmit coils 108, 108′. The cavity 117 may include a single radio-frequency transmit coil 108. The cavity 117 may include one or more, two or more, three or more, four or more, ten or less, or seven or less radio-frequency transmit coils 108.

A cross-sectional length (e.g., diameter) of the radio-frequency transmit coils 108 may be selected so that all or a portion of a patient may extend within the portable system 100. The cross-sectional length may be sufficiently large to receive an arm, a leg, a torso, two arms, two legs, a head, shoulders, hips, or a combination thereof. The radio-frequency transmit coils 108, 108′ may have a partial overlap. The radio-frequency transmit coils 108 and 108′ may be free of any overlap. The radio-frequency transmit coils 108, 108′ may be located end to end. A space may be located between ends of radio-frequency transmit coils 108 and 108′. The radio-frequency transmit coils 108 and 108′ may all be coplanar. The radio-frequency transmit coils 108 and 108′ may all be circular and may extend within a circular plane such that all of the radio-frequency transmit coils 108 and 108′ are coaxial.

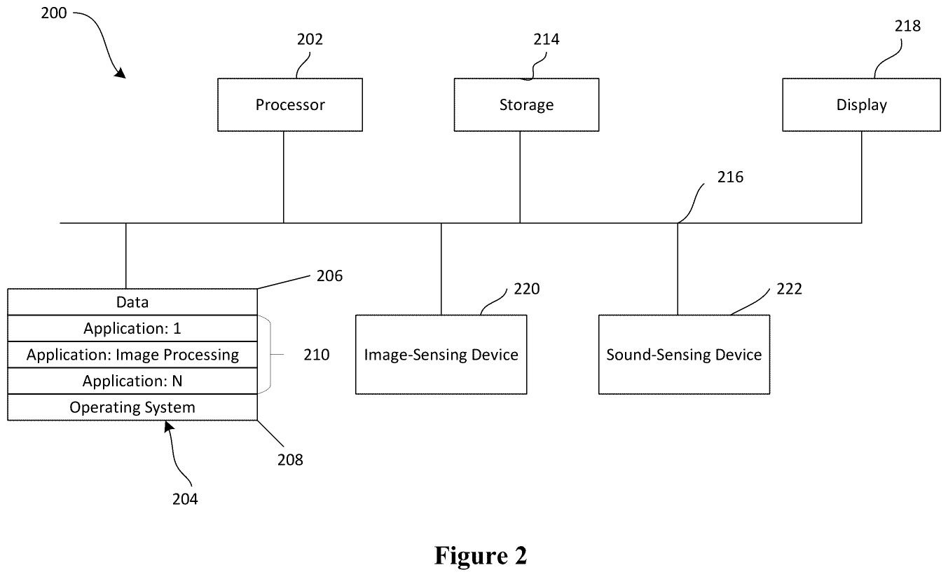

FIG. 2 is a block diagram of an example of a computing device 200. The computing device 200 can be in the form of a computing system including multiple computing devices 200, or in the form of a single computing device 200, for example, a mobile phone, a tablet computer, a laptop computer, a notebook computer, a desktop computer, the like, or a combination thereof. The computing device 200 can be communicatively connected to an MRI system, for example, to receive images from the MRI system or to control aspects of the MRI system.

A CPU 202 in the computing device 200 can be a central processing unit or any other type of device, or multiple devices, capable of manipulating or processing information now-existing or hereafter developed. Although the disclosed implementations can be practiced with a single processor as shown, e.g., the CPU 202, advantages in speed and efficiency can be achieved using more than one processor.

A memory 204 in the computing device 200 can be a read-only memory (ROM) device or a random access memory (RAM) device in an implementation. The memory 204 may be flash memory, read only memory, or both. The memory 204 can include code and data 206 that is accessed by the CPU 202 using a bus 216. The memory 204 can further include an operating system 208 and application programs 210. The application programs 210 may include at least one program that permits the CPU 202 to perform the methods described here. The computing device 200 can also include a secondary storage 214, which can, for example, be a memory card used with a computing device 200 that is mobile.

The computing device 200 may also include one or more output devices, such as a display 218. The display 218 may be, in one example, a touch sensitive display 218 that combines a display 218 with a touch sensitive element that is operable to sense touch inputs. The display 218 can be coupled to the CPU 202 via the bus 216. When the output device is or includes a display 218, the display 218 can be implemented in various ways, including by a liquid crystal display (LCD), a cathode-ray tube (CRT) display or light emitting diode (LED) display, such as an organic LED (OLED) display.

The computing device 200 can also include or be in communication with an image-sensing device 220, for example a camera, or any other image-sensing device 220 now existing or hereafter developed that can sense an image such as the image of a user operating the computing device 200.

The computing device 200 may also include or be in communication with a sound-sensing device 222, for example a microphone, or any other sound-sensing device now existing or hereafter developed that can sense sounds near the computing device 200. The sound-sensing device 222 can be positioned such that it is directed toward the user operating the computing device 200 and can be configured to receive sounds, for example, speech or other utterances, made by the user while the user operates the computing device 200.

The operations of the CPU 202 may be distributed across multiple machines (each machine having one or more of processors) that can be coupled directly or across a local area or other network. The memory 204 can be distributed across multiple machines such as a network-based memory or memory in multiple machines performing the operations of the computing device 200. The bus 216 of the computing device 200 can be composed of one or more buses 216.

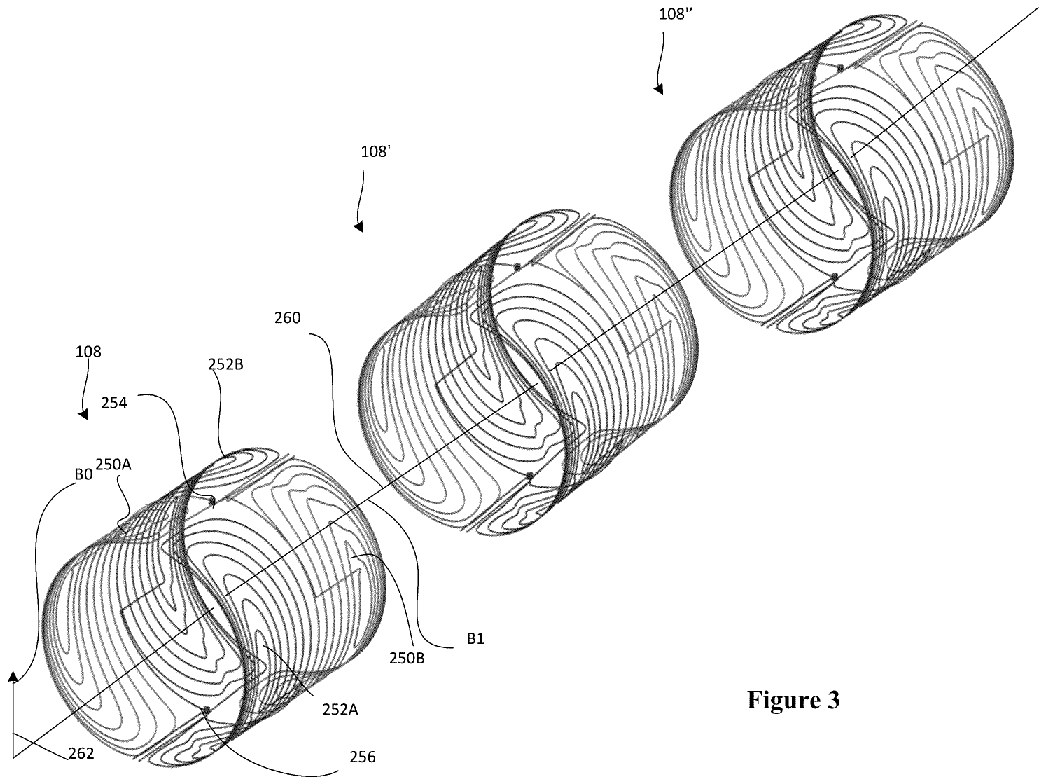

FIG. 3 illustrates an example of a series of radio-frequency transmit coils 108, 108′, and 108″ that are each a quadrature coil. The radio-frequency transmit coils 108, 108′, and 108″ may include or be worked with one or more radio-frequency reception coils 110 (RF RX coil. RF RX coil 110 may be one of the parts or devices of an MRI system 100 of FIG. 1A. Additionally, the radio-frequency transmit coils 108, 108′, and 108″ can be used as a transceiver coil which perform radio-frequency transmission and reception in the MRI system. The performance of the RF TX coil 108 may be closely associates with image quality (signal-to-noise ratio and/or contrast-to-noise ratio). The radio-frequency transmit coils 108, 108′, and 108″ that are quadrature driven may have at least one of the following functional properties: high signal-to-noise ratio, good uniformity, high unloaded quality factor (e.g., Q factor) of the resonance circuit, high transmit efficiency, reduced power deposition, and large coverage, compared to the identical radio-frequency transmit coil with a linear driven coil. The radio frequency apparatus may have an unloaded Q factor above 10, above 50, or above 100. The radio-frequency transmit coils 108, 108′, and 108″ as taught herein are quadrature coils with quadrature modes. That is, the radio-frequency transmit coils 108, 108′, and 108″ produce a circular polarization radio-frequency electromagnetic field via adjusting phases and amplitude of currents of each element of the radio-frequency transmit coils 108, 108′, and 108″. The transmit power of a quadrature coils 108, 108′, and 108″ may be ideally about 50% over a linear transmit coil. As a result, local signal to noise ratio (SNR) is greatly reduced compared to a linear transmit coil. The present radio-frequency transmit coils 108, 108′, and 108″ provide a high resolution such that MRI exams are conducted at ultra-high field strength or patients with implanted conductive metals that generate stronger eddy currents and result in increased RF power deposition. Additionally, the radio-frequency transmit coils (e.g., quadrature transmit coil) 108, 108′, and 108″ provide more uniform RF fields when compared to RF fields of a linear transmit coil. The radio-frequency transmit coils 108, 108′, and 108″ (e.g., quadrature RF coils) provide a horizontal Bi field. Both the radio transmission coils 108 (RF TC coil) and the radio transmission reception coil (RF RX coil) may be perpendicular to a Bo field. The major components of radio-frequency fields generated by the first coil resonator and the second coil resonator extend in a direction that is orthogonal and perpendicular to a main magnetic field. A combination of the first coil resonator and the second coil resonator are a radio-frequency apparatus to excite nuclear spins for the MRI and the MRS.

The radio-frequency transmit coils 108, 108′, and 108″ are a single layer coil. Thus, the radio-frequency transmit coils 108, 108′, and 108″ taught herein are a single layer quadrature coils with different resonances. The present teachings provide a novel radio-frequency quadrature transmit coil 108 for a vertical B₀ MRI system.

FIG. 3 is an example of quadrature transmit coils 108, 108′, and 108″ for a vertical Bo MRI/MRS system (spiral conductive wire) having a plurality of radio-frequency transmit coils 108, 108′, and 108″. The radio-frequency transmit coils 108, 108′, and 108″ are all identical and are all coaxial along the longitudinal axis 260. The longitudinal axis 260 of the radio-frequency transmit coils 108, 108′, and 108″ extend in a direction of the axis 260. As shown, a vertical axis 262 extends perpendicular to the longitudinal axis 260 and (B₀) extends in a same direction as the vertical axis 262. The direction of (B₁) may be along the longitudinal axis 260 as shown, but may also extend in another direction that is perpendicular to (B₀).

The radio-frequency transmit coils 108, 108′, and 108″ are quadrature transmit coils, and the transmit coil 108 includes a first coil resonator which is comprised of 250A and 250B, a second coil resonator which is comprised of 252A and 252B. The transmit coil 108 extends along the longitudinal axis 260 as transmit coil 108′ and 108″ to form a whole quadrature coil shown in FIG. 3. The first coil resonator 250A, 250B may include a plurality of conductive elements. The plurality of conductive elements may be a conductive wire that includes spiral conductive wires or multi-turn conductive wires. The conductive elements may be one or more of ground dipole coil, slot coil, dipole coil, helical coil, spiral coil, fractal coil, and microstrip coil. The second coil resonator 252A, 252B may include a plurality of conductive elements. A first coil resonator 250A is electrically separated from a second coil resonator 252A by a fist capacitive element 254 and a first coil resonator 250B is electrically separated from a second coil resonator 252B by a second capacitive element 256. Each of the coil resonators may include or be in electrical contact with one or more capacitive elements. As show, the first coil resonator 250A, 250B and the second coil resonator 252A, 252B are located within a same layer of the radio-frequency apparatus, include a same mode, are parallel to an axis of a subject being imaged, or a combination thereof. The at least one first coil resonator, the at least one second coil resonator, or both may include one or more capacitive elements, two or more capacitive elements, three or more capacitive elements, or four or more capacitive elements.

The first coil resonator 250A, 250B and the second coil resonator 252A, 252B are electromagnetically isolated relative to each other. The electromagnetic isolation may comprise one or more of capacitor decoupling, inductance decoupling, and preamplifier decoupling. The plurality of conductive elements in the first coil resonator may be identical to the plurality of conductive elements in the second coil resonator. The plurality of conductive elements in the first coil may be different from the plurality of conductive elements in the second coil resonator. An excitation mode of the first coil resonator may be identical to an excitation mode of the second coil resonator. The first coil resonator and the second coil resonator may include a same mode. The radio-frequency apparatus may generate a main field within a direction. The direction of the main field strength may be vertical or horizontal (e.g., relative to B₀). The main field may be less than 0.1 Tesla. The main field (strength) may be from 0.1 Tesla to 1.5 Tesla. The main field (strength) may be above 1.5 Tesla.

The first coil resonator and the second coil resonator may include one or more conductive elements layers along a direction of a main magnetic field. The first coil resonator and the second coil resonator may be located within a same layer. A radio transmission transmit coil configuration has a maximum magnitude of B₁⁺ and minimum magnitude of B₁⁻. A radio-frequency coil includes the at least one first coil resonator and the at least one second coil resonator and the radio-frequency coil is a plurality of radio-frequency coils that are co-axial with one another along a longitudinal axis of the radio-frequency apparatus. The plurality of radio-frequency coils are free of any overlap.

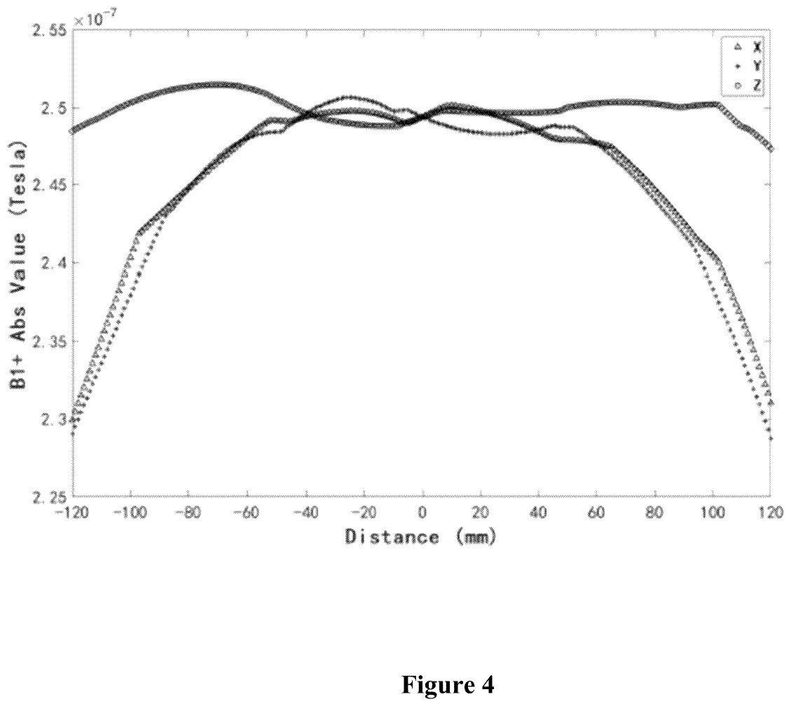

FIG. 4 illustrates a B₁⁺ magnitude of the quadrature transmit coils 108 shown in FIG. 3 along different orientations. The inhomogeneity of B₁⁺ magnitude within a spherical diameter of 20 cm is less than 1% along a z direction, 3% along both x and y directions. As shown, between 60 mm and −60 mm all three of the x, y, and z directions substantially overlap at about 0.247 micro-Tesla. This overlap demonstrates the homogeneity of the different orientations relative to 0 (e.g., a center) of the spherical diameter. As shown, the directions begin to diverge away from one another as the directions (e.g., x, y, and z) approach an outer diameter of the sphere (e.g., 120 mm and −120 mm). A magnitude of fields generated by the first coil resonator and the second coil resonator is equal to or very close to a most reasons (e.g., within about 1% or less). A phase difference between the first coil resonator and the second coil resonator may be about 90 degrees.

The present teachings provide: (1) a quadrature transmit coil in a vertical B₀ MRI system that may increase the efficiency of MRI transmission. As a result, the quadrature coils need less radio-frequency power to reach given flip angles and reduces radio-frequency power deposition because the radio-frequency power deposition is proportional to an input power from radio-frequency amplifier (when compared to a linear coil). The reduced input power leads to lower energy consumption and energy costs. The present teachings further realize (2) a single layer configuration of quadrature transmit coil with the same fundamental mode in a vertical Bo MRI system, such as LC circuit mode which frequency is equal to

$$f = \frac{1}{2 π \sqrt{LC}} ;$$

(3) using multi-turn coil or spiral coil configuration to reduce the cost (specially the cost of capacitors) and increase the efficiency of transmit field; (4) applying the configuration of two sections which are comprised of a plurality of conductive elements for quadrature driven; (5) applying litz wire to reduce the loss of transmit coil and improve the efficiency of transmit coil; and (6) easily applying the proposed configuration for parallel transmission or radio-frequency shimming at ultra-high field MRI system (>=7.0 Tesla).

The capacitance in LC circuit increases with reduced the static field strength. The lumped capacitors used for the LC circuit must be higher than the stray capacitance to avoid the shift in the resonance frequency.

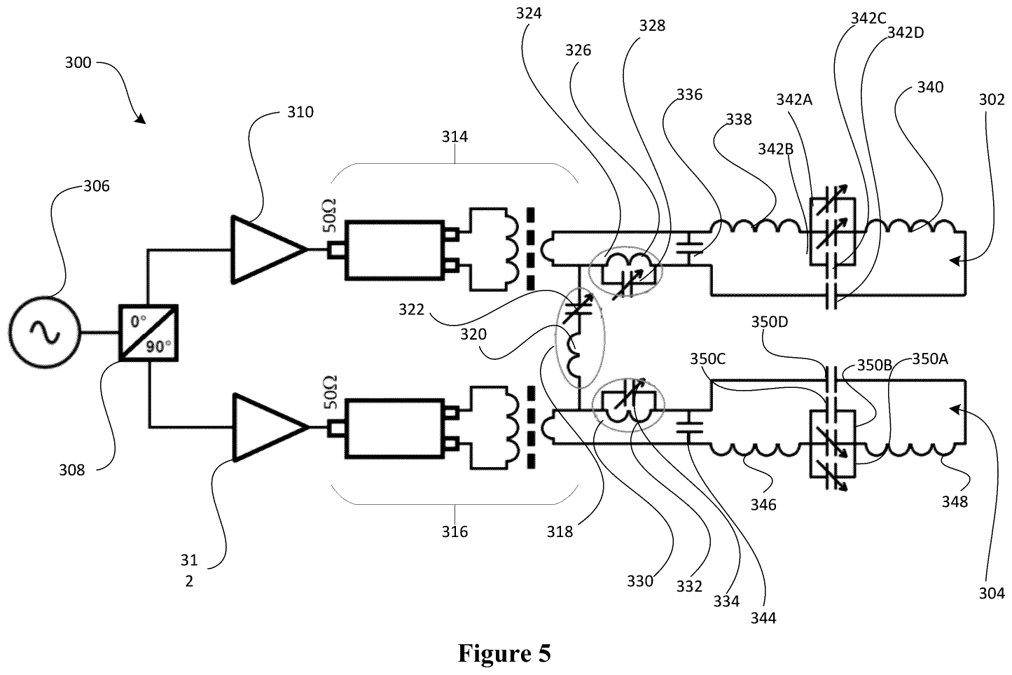

FIG. 5 illustrates a circuit 300. The circuit includes an A circuit side 302 and a B circuit side 304. The circuit 300 is connected to a transmitted RF signal source 306. The transmitted RF signal source 306 may be generate by MRI spectrometer.

The Quadrature phase shifter 308 is mainly a quadrature coupler which splits the input signal into two signals 90° out of phase. The phase shifter 308 may change the phase of the signal between the A circuit side 302 and the B circuit side 304. The phase shifter 308 may change a phase of the RF signal by 90 degrees to maximize circularly polarized RF field.

From the phase shifter 308 the RF signal source extends into a first RF power amplifier 310 on the A circuit side 302 and a second RF power amplifier 312 on the B circuit side 304. The first amplifier 310 may amplify the RF signal of low level to have an amplitude. The second amplifier 312 may amplify the signal of low level to have an amplitude. The first voltage amplitude and the second voltage amplitude, may be identical, different, have different phase. After the first voltage amplifier 310 amplifies the RF signal, the RF signal extends into a transformer, which as shown is a first balun transformer 314. After the second voltage amplifier 312 amplifies the signal, the signal extends into a transformer, which as shown is a second balun transformer 316.

The first balun transformer 314 and the second balun transformer 316 function to provide a flow of AC signals, change impedance of a voltage, balance loads of the signals, change an impedance, or a combination thereof. The first balun transformer 314, the second balun transformer 316, or both may provide a balanced output. The first balun transformers 314, the second balun transformers 316, or both may receive an unbalanced input and provide a balanced output, balance between a first side and a second side of a respective one of the first balun transformer 314 and/or the second balun transformer 316. A first side of the first balun transformers 314 and the second balun transformers 316 receives the voltage and then outputs the voltage to a second side of the first balun transformers 314 and the second balun transformers 316 respectively. The second side of the first balun transformers 314 and the second balun transformers 316 are connected by a connector LC circuit 318.

The connector LC circuit 318 includes an inductor 320 and a variable capacitor 322. The connector LC circuit 318 is used for decoupling between the A circuit 302 and the B circuit 304. The connector LC circuit 318 may act as a bandpass filter, be tunable, balance the A circuit 302 relative to the B circuit 304.

The A circuit 302 after the first balun transformer 314 may extend through an A LC circuit 324. The A LC circuit 324 that includes an inductor 326 and a variable capacitor 328 is also used for decoupling between the A circuit 302 and the B circuit 304.

After the A LC circuit 324 the voltage extends through a capacitor 336 to a first A coupled inductor 338, a second A coupled inductor 340, and a plurality of capacitors that include a first capacitor 342A (which may be a variable capacitor), a second capacitor 342B (which may be a variable capacitor), a third capacitor 342C, and a fourth capacitor 342D. The plurality of capacitors 342A-D may be connected in parallel. The plurality of capacitors 342A-D may have some static capacitors and some variable capacitors so that the voltage may be tuned, decoupled, varied, or a combination thereof.

The B circuit 304 after the second balun transformer 316 may extend through a B LC circuit 330. The B LC circuit 330 that includes an inductor 332 and a variable capacitor 334 is also used for decoupling between the A circuit 302 and the B circuit 304.

After the B LC circuit 330 the voltage extends through a capacitor 344 to a first B coupled inductor 346, a second A coupled inductor 348, and a plurality of capacitors that include a first capacitor 350A (which may be a variable capacitor), a second capacitor 350B (which may be a variable capacitor), a third capacitor 350C, and a fourth capacitor 350D. The plurality of capacitors 350A-D may be connected in parallel. The plurality of capacitors 350A-D may have some static capacitors and some variable capacitors so that the voltage may be tuned, decoupled, varied, or a combination thereof.

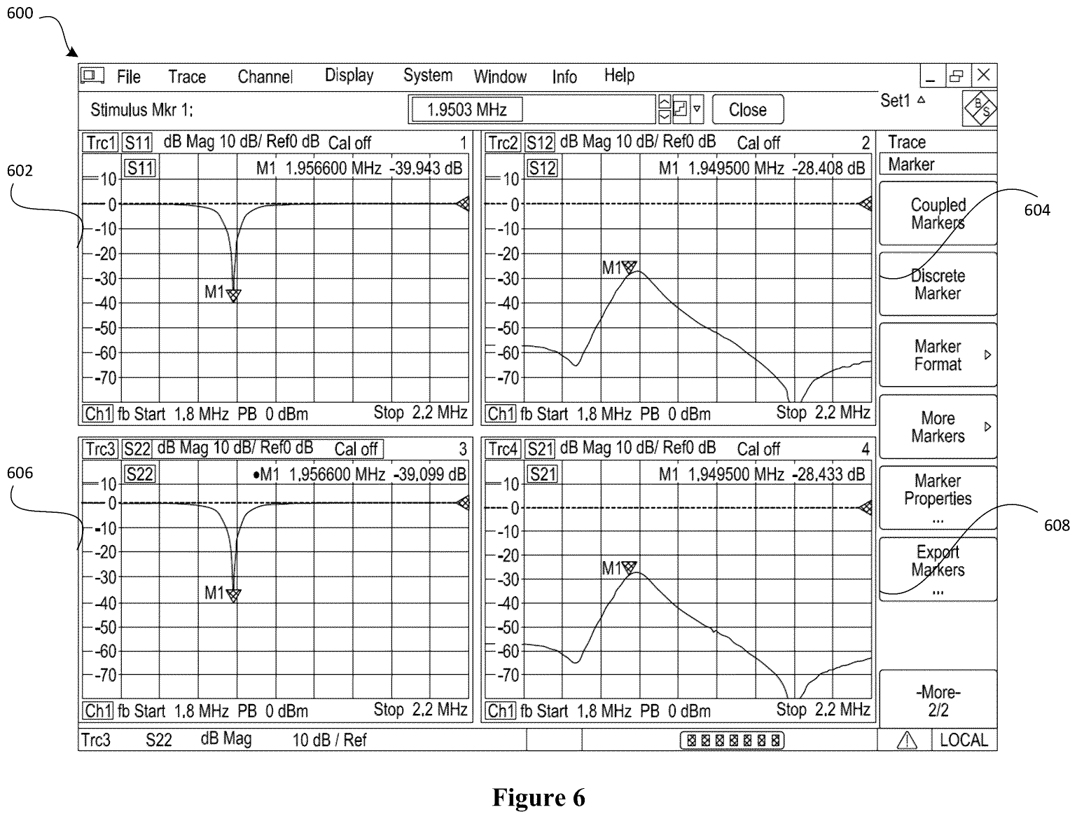

FIG. 6 illustrates a graphical representation of each channel of the quadrature RF transmit coil 600. The quadrature RF coil 600 includes a first channel 602 a second channel 604. The first channels 602 and 606 is formed by a combination of 250A and 252A of FIG. 2. The second channel 604 and 608 is formed by a combination of 250B and 252B. The graphs are formed by a network analyzer changing the channels based on a network analyzer.

Trc 1 (602) shows a reflection coefficient S 11 of a first channel 602 of the quadrature RF transmit coil 600 (e.g., the A circuit side 302). The graph demonstrates an inverse peak M1 that is measured at resonance frequency.

Trc 3 (606) shows a reflection coefficient S22 of a second channel 606 of the quadrature RF transmit coil 600 (e.g., the B circuit side 304). The inverse peak M1 is measured at resonance frequency.

Trc 2 (604) shows a reverse transfer ratio between the first channel 604 and channel 608 of the quadrature RF transmit coil 600.

Trc4 (608) shows a forward transfer ratio between the two channel 604 and channel 608 of the quadrature RF transmit coil 600.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed is:

1. A radio-frequency apparatus for magnetic resonance imaging (MRI) at least one of magnetic resonance spectroscopy (MRS) transmission, the radio-frequency apparatus comprising:
at least one first coil resonator including a plurality of conductive elements;
at least one second coil resonator including a plurality of conductive elements,
one or more of capacitive elements in each of the at least one first coil resonator and the at least one second coil resonator;
wherein the first coil resonator and the second coil resonator are located in a same layer of the radio-frequency apparatus, with a same mode, and the first coil resonator and the second coil resonator are parallel to an axis of the subject being imaged;
wherein the first coil resonator and the second coil resonator are electromagnetically isolated relative to each other;
wherein major components of radio-frequency fields generated by the first coil resonator and the second coil resonator extend in a direction that is orthogonal and perpendicular to a direction of a main magnetic field; and
wherein a combination of the first coil resonator and the second coil resonator are a radio-frequency apparatus to excite nuclear spins for the MRI and the MRS.

2. The radio-frequency apparatus in accordance with claim 1, wherein the plurality of conductive elements are comprised of one or more of ground dipole coil, slot coil, dipole coil, helical coil, spiral coil, fractal coil, and microstrip coil.

3. The radio-frequency apparatus in accordance with claim 2, wherein the plurality of conductive elements are a conductive wire that further comprises spiral conductive wires or multi-turn conductive wires.

4. The radio-frequency apparatus in accordance with claim 1, wherein electromagnetic isolation between the first coil resonator and the second coil resonator comprises one or more of capacitor decoupling, inductance decoupling, and preamplifier decoupling.

5. The radio-frequency apparatus in accordance with claim 1, wherein the plurality of conductive elements in the first coil resonator are identical to the plurality of conductive elements in the second coil resonator.

6. The radio-frequency apparatus in accordance with claim 1, wherein the plurality of conductive elements in the first coil resonator are different from the plurality of conductive elements in the second coil resonator.

7. The radio-frequency apparatus in accordance with claim 1, wherein an excitation mode of the first coil resonator is identical to an excitation mode in the second coil resonator.

8. The radio-frequency apparatus in accordance with claim 1, wherein magnitude of Bi+fields generated by the first coil resonator and the second coil resonator is equal to or very close at most regions.

9. The radio-frequency apparatus in accordance with claim 1, wherein the direction of main field strength is either vertical or horizontal, a phase difference between the first coil resonator and the second coil resonator is 90°, or both.

10. The radio-frequency apparatus in accordance with claim 1, wherein the main magnetic field strength is less than 0.1 Tesla

11. The radio-frequency apparatus in accordance with claim 1, wherein the main magnetic field strength is from 0.1 Tesla to 1.5 Tesla.

12. The radio-frequency apparatus in accordance with claim 1, wherein the main magnetic field strength is above 1.5 Tesla.

13. The radio-frequency apparatus in accordance with claim 1, wherein both the first coil resonator and the second coil resonator include one or more of conductive element layers along a direction of main magnetic field.

14. The radio-frequency apparatus in accordance with claim 1, wherein the radio-frequency apparatus for transmission has a maximum magnitude of B₁⁺ and minimum magnitude of B c.

15. The radio-frequency apparatus in accordance with claim 1, wherein an unloaded Q factor of the radio-frequency apparatus is above 50.

16. The radio-frequency apparatus in accordance with claim 1, wherein an unloaded Q factor of the radio-frequency apparatus is above 100.

17. The radio-frequency apparatus in accordance with claim 1, wherein the at least one first coil resonator and the at least one second coil resonator are comprised of a radio-frequency coil and the radio-frequency coil is a plurality of radio-frequency coils that are co-axial with one another along a longitudinal axis of the radio-frequency apparatus.

18. The radio-frequency apparatus in accordance with claim 17, wherein the plurality of radio-frequency coils are free of any overlap.

19. A magnetic resonance imaging (MRI), the MRI comprising a radio-frequency apparatus, the radio-frequency apparatus comprising:
at least one first coil resonator including a plurality of conductive elements;
at least one second coil resonator including a plurality of conductive elements,
one or more of capacitive elements in each of the at least one first coil resonator and the at least one second coil resonator;
wherein the first coil resonator and the second coil resonator are located in a same layer of the radio-frequency apparatus, with a same mode, and the first coil resonator and the second coil resonator are parallel to an axis of the subject being imaged;
wherein the first coil resonator and the second coil resonator are electromagnetically isolated relative to each other;
wherein major components of radio-frequency fields generated by the first coil resonator and the second coil resonator extend in a direction that is orthogonal and perpendicular to a main magnetic field; and
wherein a combination of the first coil resonator and the second coil resonator are a radio-frequency apparatus to excite nuclear spins for the MRI.

20. A magnetic resonance spectroscopy (MRS), the MRS comprising a radio-frequency apparatus, the radio-frequency apparatus comprising:
at least one first coil resonator including a plurality of conductive elements;
at least one second coil resonator including a plurality of conductive elements,
one or more of capacitive elements in each of the at least one first coil resonator and the at least one second coil resonator;
wherein the first coil resonator and the second coil resonator are located in a same layer of the radio-frequency apparatus, with a same mode, and the first coil resonator and the second coil resonator are parallel to an axis of the subject being imaged;
wherein the first coil resonator and the second coil resonator are electromagnetically isolated relative to each other;
wherein major components of radio-frequency fields generated by the first coil resonator and the second coil resonator extend in a direction that is orthogonal and perpendicular to a main magnetic field; and
wherein a combination of the first coil resonator and the second coil resonator are a radio-frequency apparatus to excite nuclear spins for the MRS.
